(19) United States
(12) Patent Application Publication
(10) Pub. No.: US 2012/0323259 A1
(43) Pub. Date: Dec. 20, 2012

(54) KIT AND METHOD FOR EXTRACTING AND STORING A SKIN TISSUE SAMPLE

(76) Inventor: WILL RICHARDSON, FORT LAUDERDALE, FL (US)

(21) Appl. No.: 13/163,073

(22) Filed: Jun. 17, 2011

(51) Int. Cl. A61B 17/50

(52) U.S. Cl. 606/132

(57) ABSTRACT

A kit of components for extracting and storing a skin tissue sample includes an extraction cartridge with a plurality of blades, an actuator cartridge, and a cap with an aqueous preservative. The actuator cartridge is removeably attached to the extraction cartridge and causes the plurality of blades of the extraction cartridge to extract a skin tissue sample from the person. After the skin tissue sample is extracted, the cap is attached to the extraction cartridge to store and suspend the skin tissue sample in an aqueous preservative preloaded in a container in the cap. A liquid-tight seal prevents bodily fluids from contaminating the actuator cartridge and this enables the actuator cartridge to be removed from the extraction cartridge and to be reused. The extraction cartridge and the attached cap, which store the skin tissue sample, are sent to medical laboratory so that the skin tissue sample can be analyzed.

### BACKGROUND OF THE INVENTION

1. Field of the Invention

The invention relates to a kit of components and to a method of using the components to enable any person to easily extract a skin tissue sample and then store the skin tissue sample in a manner such that the skin tissue sample may be sent to a medical laboratory for analysis.

2. Description of the Related Art

When a person has a suspicious skin growth, for example, a mole that has changed in size, that person must make an appointment with a specialized medical professional, such as a dermatologist, who has special training in extracting skin samples of suspicious skin growths. This medical professional will typically extract a skin sample from each suspicious skin growth on the person and send the samples to a medical laboratory for analysis. Unfortunately, when that person contacts the medical professional to make an appointment, there can be a significant amount of time, for example, a few weeks or more, until that person is able to see that medical professional at the set appointment time. During the delay between the time the appointment is made and the time the person sees the medical professional at the scheduled appointment time, the suspicious skin growth can increase in size and become more of a problem. If the suspicious skin growth is dealt with at an early time, the probability is greater that it will be a minor issue. Therefore, it is desirable to minimize the amount of delay between the time the person makes the appointment and the time the person sees the medical professional, such as a dermatologist, who has special training in extracting skin samples of suspicious skin growths.

### BRIEF SUMMARY OF THE INVENTION

However, in contrast to the traditional approach, the invention is not focused on the goal of minimizing the amount of delay between the time the person makes the appointment and the time the person sees a specialized medical professional who has the conventional special training in extracting skin samples of suspicious skin growths. Rather, it is an object of the invention to totally eliminate the need for this specialized medical professional who has the conventional special training to perform the procedure of extracting skin samples of suspicious skin growths.

The invention is directed towards an untrained person that does not have the conventional special training in extracting skin samples of suspicious skin growths. It is an object of the invention to enable such an untrained person to extract skin samples of suspicious skin growths.

With the foregoing and other objects in view, the invention enables a person to easily extract skin tissue samples from suspicious skin growths on that person's own body. This eliminates the need for that person to visit any type of medical professional for the purpose of having the medical professional extract the skin tissue samples. The invention enables people to obtain skin tissue samples much quicker and easier, and it is also helpful to people residing or working in rural or remote areas where convenient access to medical professionals is limited or nonexistent.

It is an another object of the invention to enable a family doctor or some other medical professional, who does not have a lot of training in the conventional ways of extracting skin samples, to easily extract skin tissue samples from suspicious skin growths on the body of a patient. The meaning of “conventional ways of extracting skin samples” means the ways that are commonly used at the time of the present invention. This option enables a person, who is concerned about a suspicious skin growth on their body, to see their trusted family physician and have their family physician use the invention to extract the skin tissue sample. The person can likely visit their family physician more quickly when compared with the delay required to see a specialist, such as a dermatologist. The visit to the family physician will also likely cost less when compared with the cost of visiting a specialist, such as a dermatologist.

It is also object of the invention to enable a relatively untrained medical professional, such as a medical assistant, to easily extract skin tissue samples from suspicious skin growths on the body of a patient. By providing a way for a relatively untrained medical professional to extract skin tissue samples, the cost of the procedure is even further reduced. Also, since the person or patient does not have to wait to see a specialist or any type of physician, an appointment can be obtained much more quickly.

By totally eliminating the need to see any type of medical professional, people can get one or more skin samples extracted quicker and easier and can provide the sample(s) to a medical laboratory for analysis at an earlier time. Even when the person prefers to see some type of medical professional, the invention enables the extraction to be performed at an earlier time since the appointment time will typically be much earlier when compared to the typical wait time to visit a specialist, such as a dermatologist.

With the foregoing and other objects in view there is provided, in accordance with the invention, a kit of components for extracting a skin tissue sample and then storing the skin tissue sample in a manner such that the skin tissue sample may be sent to a medical laboratory for analysis. There is also provided a method of using the components for extracting a skin tissue sample and then storing the skin tissue sample. It should be understood that the invention covers not only the objects discussed above, but also covers additional objects discussed later in this document, as well as other objects that will be apparent to one of ordinary skill in the art after reading the disclosure herein.

With the foregoing and other objects in view there is provided, in accordance with the invention, a kit of components for extracting a skin tissue sample and for subsequently storing the skin tissue sample. The kit includes a reusable actuator cartridge that has a housing with a first end and a second end. The second end of the actuator cartridge has a connector. The housing of the actuator cartridge includes an actuator with a hammer moveable towards and away from the first end of the actuator cartridge. The kit also includes an extraction cartridge with a housing that has a first end. The first end of the housing includes a connector that is removeably connectable to the connector at the second end of the actuator cartridge. The extraction cartridge includes a carrier with a plurality of blades. The extraction cartridge includes a second end with a connector. The carrier is moveably disposed within the housing of the extraction cartridge to move the plurality of blades outside of the housing of the extraction cartridge for extracting a skin tissue sample. The housing of the extraction cartridge includes a liquid-tight seal disposed to isolate the plurality of blades from the first end of the extraction cartridge. The kit may also include a cap that has a container preloaded with an aqueous preservative for storing the skin tissue sample. The cap includes a connector that mates with the connector at the second end of the extraction cartridge.

With the foregoing and other objects in view there is provided, in accordance with the invention, a method for extracting and storing a skin tissue sample. The method includes a step of: placing an extraction cartridge against a portion of skin of a person and actuating an actuator cartridge that is removeably attached to the extraction cartridge to cause a plurality of blades of the extraction cartridge to extract a skin tissue sample from the person. The method also includes a step of: attaching a cap to the extraction cartridge to suspend and store the skin tissue sample in an aqueous preservative preloaded in a container in the cap.

Other features which are considered as characteristic for the invention are set forth in the appended claims.

Although the invention is illustrated and described herein as embodied in a kit of components for extracting and storing a skin tissue sample and in a method for extracting and storing a skin tissue sample, it is nevertheless not intended to be limited to the details shown, since various modifications and structural changes may be made therein without departing from the spirit of the invention and within the scope and range of equivalents of the claims.

The construction of the invention, however, together with additional objects and advantages thereof will be best understood from the following description of the specific embodiment when read in connection with the accompanying drawings.

### BRIEF DESCRIPTION OF THE SEVERAL VIEWS OF THE DRAWING

FIG. 1 is a diagram of a kit of components for extracting a skin tissue sample;

FIG. 2 is a cross-sectional view of an exemplary embodiment of the reusable actuator cartridge;

FIG. 3 is a perspective view of the reusable actuator cartridge;

FIG. 4 is a cross-sectional view of an exemplary embodiment of an extraction cartridge; and

FIG. 5 is a cross-sectional view showing a portion of the extraction cartridge, a portion of the protective shield of the reusable actuator cartridge, and a cap.

### DESCRIPTION OF THE PREFERRED EMBODIMENTS OF THE INVENTION

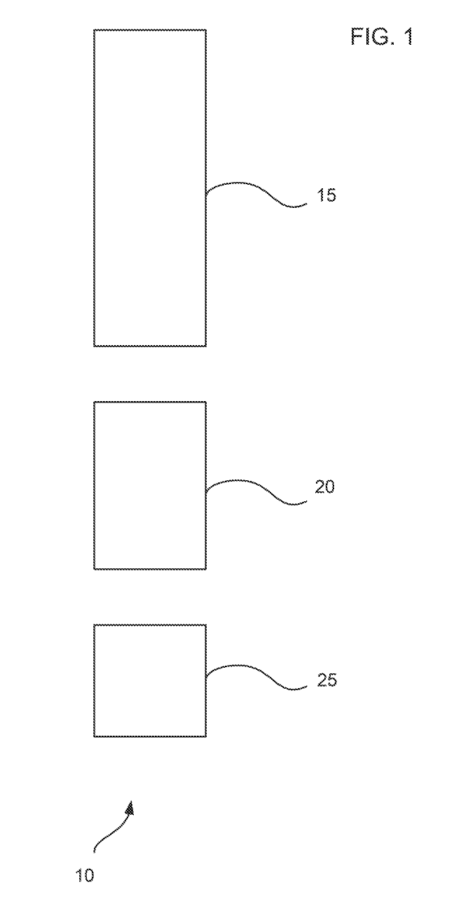

Referring now to the figures of the drawing in detail and first, particularly, to FIG. 1 thereof, there is shown a diagram of a kit 10 of components for extracting and storing a skin tissue sample. The kit 10 includes a reusable actuator cartridge 15, an extraction cartridge 20, and a cap 25. The extraction cartridge 20 is connected to the actuator cartridge 15 in order to extract a skin tissue sample. After the skin tissue sample has been extracted, the cap 25 is connected to the actuator cartridge 15 so that the skin tissue sample is suspended in a preservative stored in the cap 25. After the cap 25 has been connected to the extraction cartridge 20, the actuator cartridge 15 is preferably disconnected from the extraction cartridge 20 so that, if necessary, the actuator cartridge 15 can be used again with a different extraction cartridge to obtain another skin tissue sample. The cap 25 and the extraction cartridge 20 form a liquid-tight container that holds the skin tissue sample and that is used to transport the skin tissue sample to a medical laboratory. Since the actuator cartridge 15 is no longer attached, the size and weight of the transported unit, namely, the cap 25 and the extraction cartridge 20, is less than if the actuator cartridge 15 were still attached.

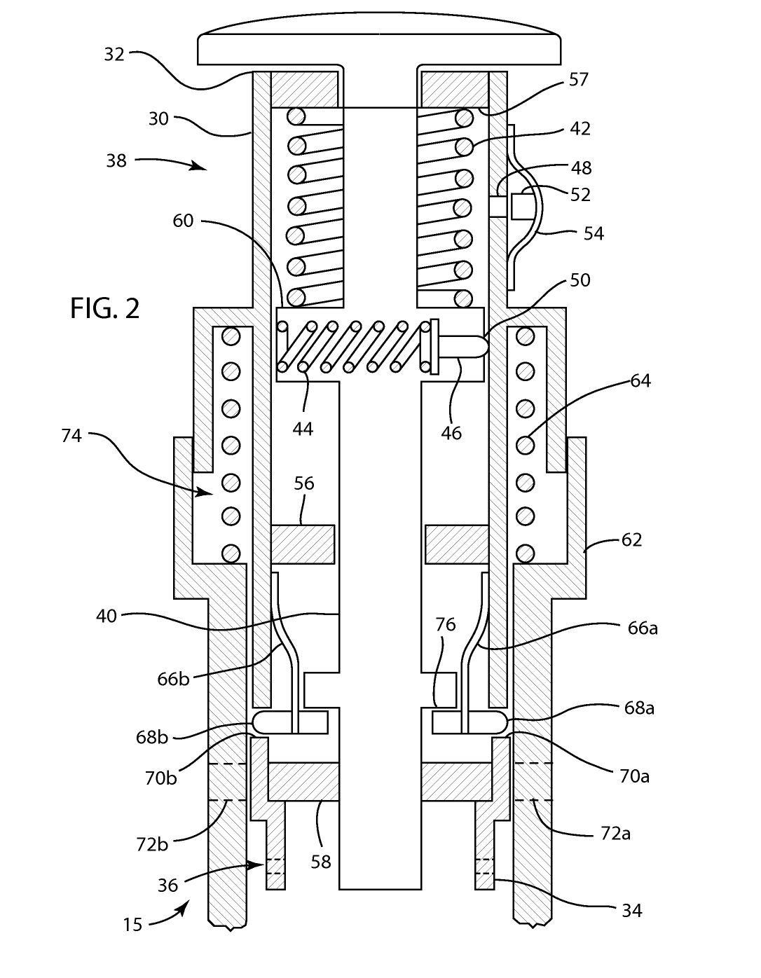

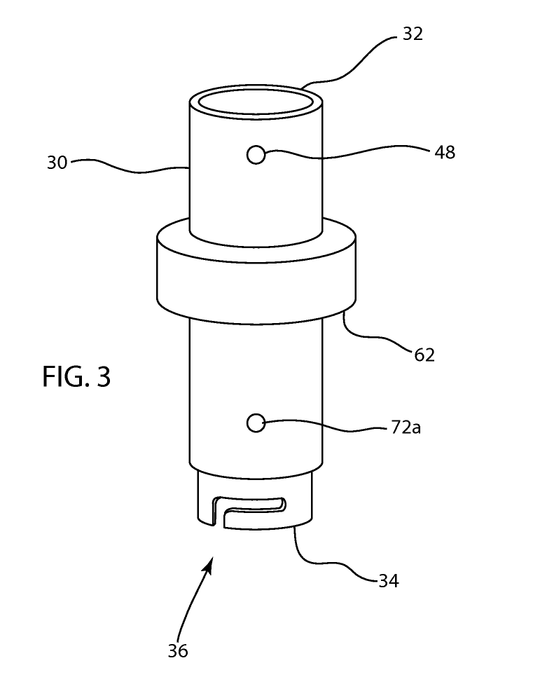

FIG. 2 is a cross-sectional view of an exemplary embodiment of a reusable actuator cartridge 15. The actuator cartridge 15 includes a housing 30 with a first end 32 and a second end 34. The second end 34 of the actuator cartridge 15 has a connector 36. FIG. 3 is a perspective view of the actuator cartridge 15. The connector 36 is seen more clearly in FIG. 3. The connector 36 can be constructed as a slot formed near the second end 34 of the actuator cartridge 15. The connector 34, which is formed as the slot, is constructed to be removeably connected or mated with a connector constructed as a pair of pins formed on the extraction cartridge 20 (not shown in FIG. 3).

Referring again to FIG. 2, the housing 30 of the actuator cartridge 15 includes an actuator 38 with a hammer 40 that is disposed to move towards and away from the first end 32 of the actuator cartridge 15. Several guides 56, 57, 58 are disposed inside the housing 30 to guide the movement of the hammer 40 within the housing 30. The actuator 38 includes a releasable spring 42 that is biased to urge the hammer 40 away from the first end 32 of the actuator cartridge 15. A latch assembly serves to hold the actuator 38 in a state in which the spring 42 is compressed between a surface of the stationary guide 57 and a surface 60 of the hammer 40. The latch assembly includes a spring 44, a pin 46, and two holes 48, 50. The hammer 40 holds the spring 44 that urges the pin 46 through the hole 50 formed in the wall of the hammer 40. When the hammer 40 is manually pushed toward the first end 32 of the actuator cartridge 15, the spring 42 is compressed until the pin 46 is aligned with and enters the hole 48 formed in the housing 30 of the actuator cartridge 15. While the pin 46 is in the hole 48, the hammer 40 is kept from moving and the actuator 38 cannot be released. In this state, the latch assembly prevents the actuator 38 from actuating the extraction cartridge 20 when the extraction cartridge 20 and the actuator cartridge 15 are connected together.

The actuator 38 includes a releasing mechanism that could be formed by a flexible membrane 54 and a pin 52. The flexible membrane 54 has ends attached to the outer wall of the housing 30. The flexible membrane 54 is attached to a pin 52. When the flexible membrane 54 holds the pin 52 outside of the hole 48, the pin 46 can securely rest in the hole 48 and hold the spring 42 in the compressed state. However, when the person operating the actuator cartridge 15, presses the flexible membrane 54 such that the pin 52 enters the hole 48, the pin 52 will push the pin 50 out of the hole 48 and the spring 42 will be released to urge or push the hammer 40 away from the first end 32 of the actuator cartridge 15. It should be clear that when the pin 46 is forced out of the hole 48, the hammer 40 of the actuator 38 is released and it will act on components of the extraction cartridge 20 to actuate the extraction cartridge 20. This will be described further below.

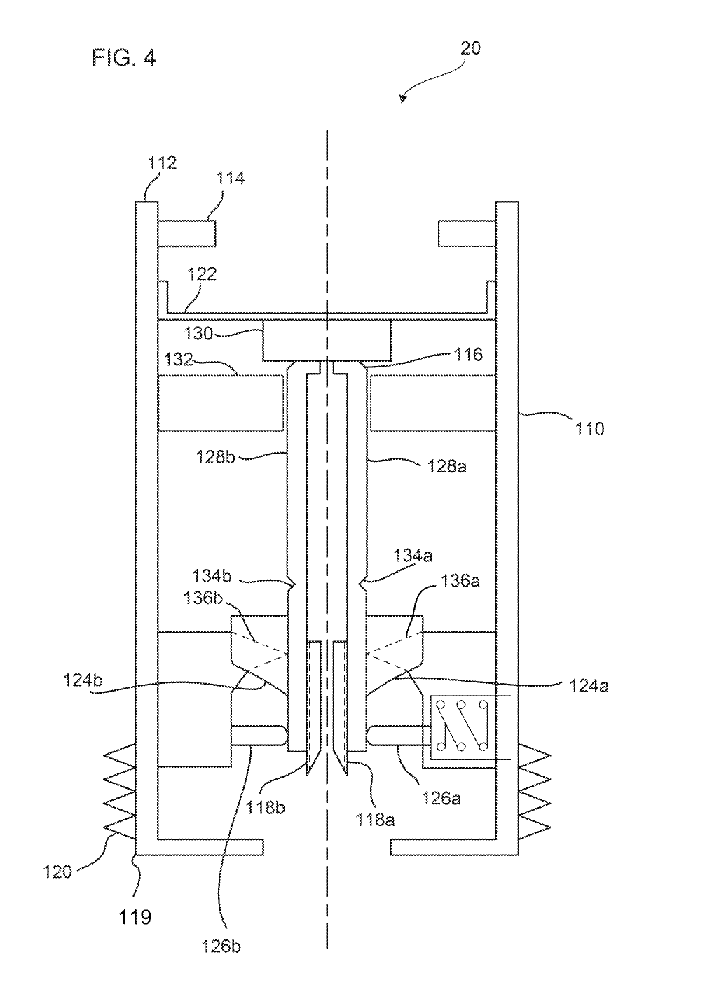

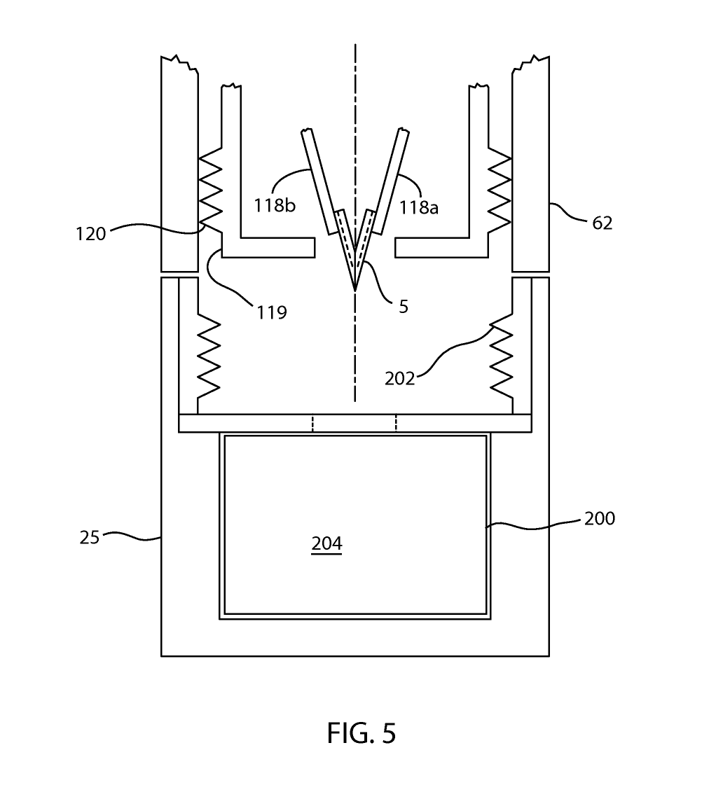

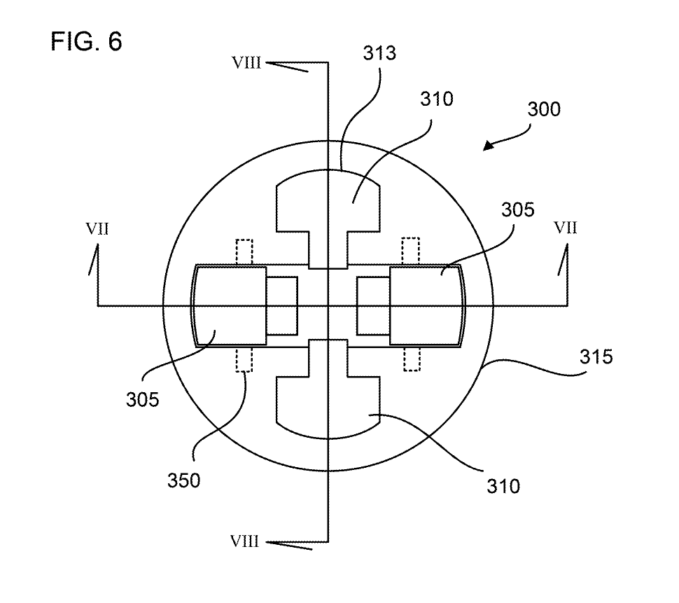

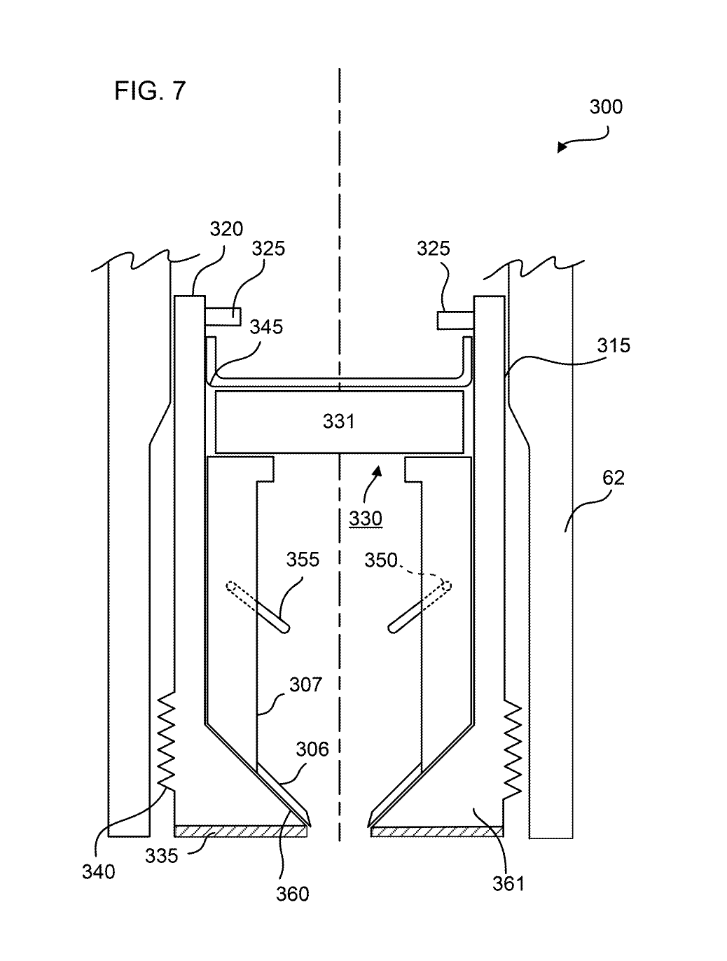

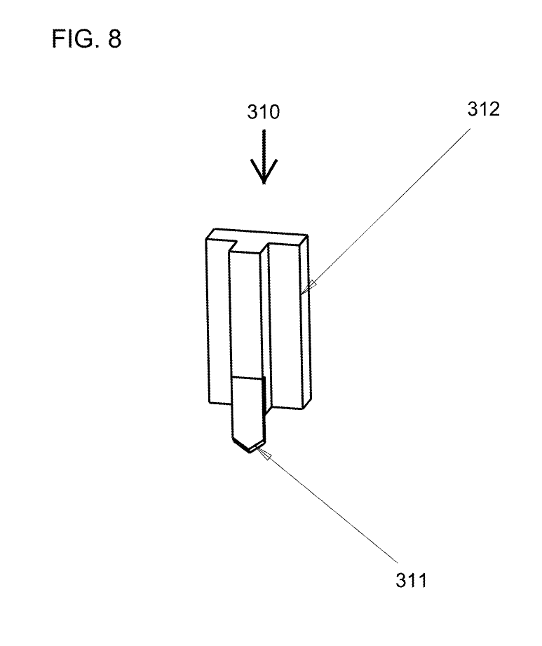

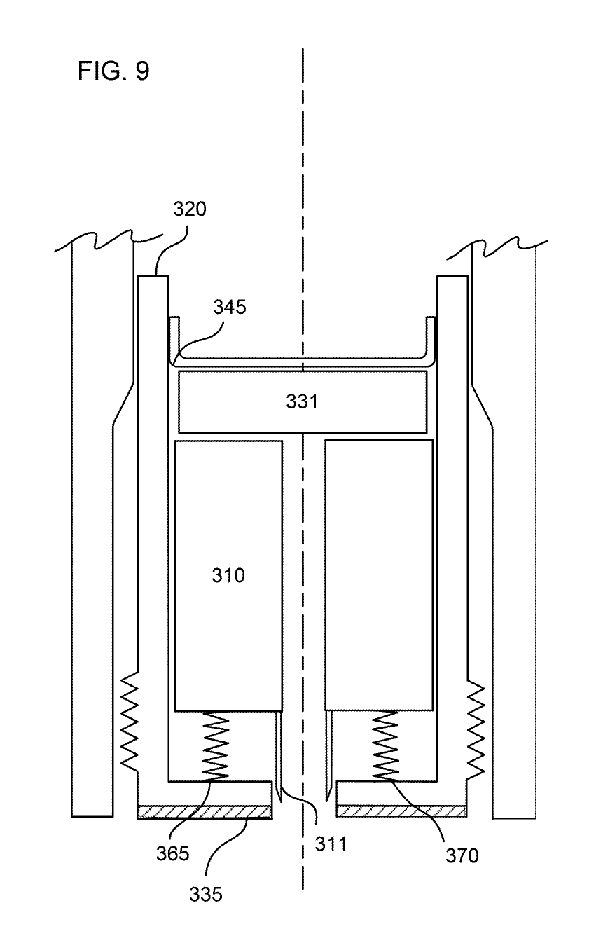

The actuator cartridge 15 may also include a protective device serving to prevent the hammer 40 from being fully actuated unintentionally. One exemplary embodiment of such a protective device includes a protective shield 62 that is moveable to a protective position in which the plurality of blades 118a, 118b (See FIG. 5) of an extraction cartridge 20 (See FIGS. 4 and 5) that is connected to the actuator cartridge 15 cannot move past the protective shield 62 of the actuator cartridge 15 and are thereby prevented from extracting a skin tissue sample. The exemplary embodiment of the protective device also includes a shield spring 64, a pair of leaf springs 66A, 66B, a pair of pins 68A, 68B, a pair of holes 70A, 70B formed in the housing 30 of the actuator cartridge 15, and a pair of holes 72A, 72B formed in the protective shield 62. The shield spring 64 is held in a cavity 74 formed between the housing 30 and the protective shield 62. When the protective shield 62 is in the protective position, the pins 68A, 68B are positioned to extend into the interior of the housing 30 and serve to limit movement of the hammer 40 when the stopping surface 76 of the hammer 40 meets the pins 68A, 68B. However, the protective shield 62 can be moved into a non-protective position by moving the protective shield 62 against the force of the shield spring 64 such that the pins 68A, 68B pass through the holes 70A, 70B formed in the housing 30 and into the holes 72A, 72B formed in the protective shield 62. In this non-protective position, the pins 68A, 68B no longer extend far enough into the interior of the housing 30 to meet the stopping surface 76 of the hammer 40. The stopping surface 76 of the hammer 40 can now move freely past the pins 68A, 68B in an amount that is sufficient to actuate the extraction cartridge 20 in manner such that the skin tissue sample is extracted. The outer ends of the pins 68A, 68B are chamfered and the leaf springs 66A, 66B are constructed with a spring constant enabling the pins 68A, 68B to be released from the holes 72A, 72B so that the protective shield 62 can be moved back to the protective position after the hammer 40 has been fully actuated.

FIG. 3 is a perspective view of the actuator cartridge 15 in which the outer wall of the housing 30, with the hole 48 formed therein, and the outer wall of the protective shield 62, with the hole 72A formed therein, can be seen. The hammer 40 is not shown for clarity.

FIG. 4 is a cross-sectional view of an exemplary embodiment of an extraction cartridge 20. The extraction cartridge 20 is intended to be used once to obtain a single skin tissue sample. The extraction cartridge 20 includes a housing 110 with a first end 112 having a connector 114 in the form of pins that are removeably connectable to the connector 36 (See FIG. 3) formed by the slots at the second end of the actuator cartridge 15. It can be seen that the pins of the connector 114 are slid into the slots forming the connector 36 of the actuator cartridge 15 (See FIG. 3).

FIG. 4 also shows that the extraction cartridge 20 includes a carrier 116 with a plurality of blades 118A, 118B. Although only two blades 118A, 118B are shown in this embodiment, it should be understood that four or even more blades could also be used. The extraction cartridge 20 includes a second end 119 having a connector 120. The connector 120 may be formed as threads on the outer surface of the housing 110. The carrier 116 is moveably disposed within the housing 110 of the extraction cartridge 20 to move the plurality of blades 118A, 118B outside of the extraction cartridge 20 for extracting a skin tissue sample. The housing 110 of the extraction cartridge 20 includes a liquid-tight seal 122 disposed to isolate the plurality of blades 118A, 118B from the first end 112 of the extraction cartridge 20. The liquid-tight seal 122 is preferably a flexible membrane. The liquid-tight seal 122 prevents any bodily fluids from getting on the hammer 40, the housing 30 or any internal parts of the actuator cartridge 15. This enables the actuator cartridge 15 to be reused without the danger of contaminating another skin tissue sample that will be extracted using a different extraction cartridge.

FIG. 4 shows that a pair of cams 124A, 1248 is formed on the carrier 116. The carrier 116 includes two legs 128A, 1288 that are pivotally mounted by a hinge mechanism attached near the liquid-tight seal 122. As the hammer 40 (not shown in FIG. 4) moves away from the first end 32 of the actuator cartridge 15, the hammer 40 pushes against the liquid-tight seal 122 and the spacer 130 and moves the carrier 116. As the carrier 116 moves through the guide 132 and moves away from the first end 112 of the extraction cartridge 20, the cams 124A, 1248 move past the cam followers 126A, 1268, which causes the blades 118A, 1188 to move inward, as they also move outside of the extraction cartridge 20 to extract a skin tissue sample 5 (See FIG. 5). Thus, the blades 118A, 118B cut and pinch the skin tissue sample 5. The cam followers 126A, 126B may be constructed as spring loaded pins that are urged towards the interior of the housing 110. Fulcrums 136A, 1368 extend from the inner surface of the housing 110. When the blades 118A, 1188 have been moved past the end 119 of the extraction cartridge 20 to penetrate the skin by approximately 4 mm, the movement of the blades 118A, 1188 is stopped when the fulcrums 136A, 1368 engage the notches 134A, 1348 formed in the legs 128A, 1288 of the carrier 116. When the fulcrums 136A, 1368 engage the notches 134A, 1348, the blades 118A, 1188 are locked in a position extending outside of the extraction cartridge 20, and the blades 118A, 1188 cannot be moved back inside the extraction cartridge 20 to be reused for extracting another skin sample.

FIG. 5 is a cross-sectional view showing a portion of the extraction cartridge 20, a portion of the protective shield 62 of the actuator cartridge 15, and a cap 25. FIG. 5 shows the extraction cartridge 20 after the blades 118A, 1188 have extracted a skin tissue sample 5. The cap 25 includes a container 200 that may be a heat sealed pouch. The container 200 is preloaded with an aqueous preservative 204, such as formalin, for storing the skin tissue sample 5. The cap 25 includes a connector 202 that mates with the connector 120 at the second end 119 of the extraction cartridge 20. In the exemplary embodiment, the connector 202 is formed as threads that mate with the threads forming the connector 120 of the extraction cartridge 20. FIG. 5 shows the state in which the protective shield 62 will be moved back to expose the connector 120 of the extraction cartridge 20 so that the connector 202 of the cap 25 can be attached to the connector 120 of the extraction cartridge 20. When the cap 25 is attached to the extraction cartridge 20, the blades 118A, 1188 pierce the container 200 so that the skin tissue sample 5 is held in the aqueous preservative 204. The actuator cartridge 15 can then be removed from the extraction cartridge 20. Then, the cap 25 and the extraction cartridge 20 can be sent to a medical laboratory so that the skin tissue sample 5 can be examined and analyzed.

I claim:

1. A kit of components for extracting and storing a skin tissue sample, comprising:
a reusable actuator cartridge including a housing with a first end and a second end, said second end of said actuator cartridge having a connector, said housing of said actuator cartridge including an actuator with a hammer moveable towards and away from said first end of said actuator cartridge;
an extraction cartridge including a housing with a first end having a connector being removeably connectable to said connector at said second end of said actuator cartridge, said extraction cartridge including a carrier with a plurality of blades, said extraction cartridge including a second end having a connector, said carrier being moveably disposed within said extraction cartridge to move said plurality of blades outside of said extraction cartridge for extracting a skin tissue sample, said housing of said extraction cartridge including a liquid-tight seal disposed to isolate said plurality of blades from said first end of said extraction cartridge; and
a cap including a container preloaded with an aqueous preservative for storing the skin tissue sample, said cap including a connector that mates with said connector at said second end of said extraction cartridge.

2. The kit according to claim 1, wherein:
said connector at said first end of said extraction cartridge is connected to said connector at said end of said actuator cartridge; and
as said hammer moves away from said first end of said actuator cartridge, said hammer moves said carrier to thereby move said plurality of blades outside of said extraction cartridge to extract the skin tissue sample.

3. The kit according to claim 2, wherein said seal is disposed between said plurality of blades of said extraction cartridge and said hammer of said actuator cartridge.

4. The kit according to claim 3, wherein said seal is a flexible membrane.

5. The kit according to claim 2, wherein said actuator cartridge includes a protective shield that extends past said second end of said actuator cartridge, said protective shield is moveably mounted to said housing of said actuator cartridge.

6. The kit according to claim 1, wherein said actuator cartridge includes a protective shield that extends past said second end of said actuator cartridge, said protective shield is moveably mounted to said housing of said actuator cartridge.

7. The kit according to claim 1, wherein said cap includes a heat sealed pouch that is preloaded with said aqueous preservative.

8. The kit according to claim 1, wherein said actuator cartridge includes a releasable spring that is biased to urge said hammer away from said first end of said actuator cartridge when released.

9. The kit according to claim 8, wherein said actuator cartridge includes a releasable spring that is biased to urge said actuator towards said first end of said actuator cartridge to reset the actuator.

10. The kit according to claim 1, wherein said second end of said extraction cartridge has a surface with an alginate disposed thereon.

11. The kit according to claim 1, further comprising at least one fulcrum extending from said housing of said extraction cartridge and at least one notch formed in said carrier of said extraction cartridge, said fulcrum engaging said notch to lock said plurality of blades in a position extending outside of said extraction cartridge.

12. A method for extracting and storing a skin tissue sample, which comprises:
placing an extraction cartridge against a portion of skin of a person and actuating an actuator cartridge that is removeably attached to the extraction cartridge to cause a plurality of blades of the extraction cartridge to extract a skin tissue sample from the person; and
attaching a cap to the extraction cartridge to suspend and store the skin tissue sample in an aqueous preservative preloaded in a container in the cap.

13. The method according to claim 12, which comprises removing the actuator cartridge from the extraction cartridge.

14. The method according to claim 13, which comprises sending the cap and the extraction cartridge to a medical laboratory.

15. The method according to claim 12, wherein the steps of actuating the actuator cartridge and attaching the cap to the extraction cartridge are not performed in a medical facility.

16. The method according to claim 12, wherein the steps of actuating the actuator cartridge and attaching the cap to the extraction cartridge are performed by a person that is not a medical professional.

17. The method according to claim 12, which comprises:
removing the actuator cartridge from the extraction cartridge; and
attaching another extraction cartridge to the actuator cartridge and actuating the actuator cartridge to obtain another skin tissue sample from the person.

18. The method according to claim 17, which comprises before performing the step of attaching another extraction cartridge to the actuator cartridge: resetting an actuator of the actuator cartridge.

19. The method according to claim 12, which comprises: before performing the step of placing the extraction cartridge against the portion of skin of the person, moving a protective shield from a protective position in which the plurality of blades cannot be actuated to a non-protective position in which the plurality of blades can be actuated to obtain the skin tissue sample.

20. The method according to claim 12, which comprises: unlocking a latch so that a spring and a hammer of the actuator cartridge cause a plurality of blades of the extraction cartridge to extract the skin tissue sample from the person.
